SEQUENCE LISTING (12 sequences)

SEQ ID NO: 1 - protein, 298 aa, Homo sapiens
MARRSRHRLLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLNISGIIAAVVVVALVISVCGLGVCYAQRKGYFSKETSFQKSNSSSKATTMSENDFKHTKSFII

SEQ ID NO: 2 - protein, 323 aa, Homo sapiens
MARRSRHRLLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLNISGIIAAVVVVALVISVCGLGVCYAQRKGYFSKETSFQKSNSSSKATTMSENVQWLTPVIPALWKAAAGGSRGFQAHKILYNLKTPL

SEQ ID NO: 3 - protein, 310 aa, Homo sapiens
MALRRPPRLRLCARLPDFFLLLLFRGCLIGAVNLKSSNRTPVVQEFESVELSCIITDSQTSDPRIEWKKIQDEQTTYVFFDNKIQGDLAGRAEILGKTSLKIWNVTRRDSALYRCEVVARNDRKEIDEIVIELTVQVKPVTPVCRVPKAVPVGKMATLHCQESEGHPRPHYSWYRNDVPLPTDSRANPRFRNSSSHLNSETGTLVFTAVHKDDSGQYYCIASNDAGSARCEEQEMEVYDLNIGGIIGGVLVVLAVLALITLGICCAYRRGYFINNKQDGESYKNPGKPDGVNYIRTDEEGDFRHKSSFVI

SEQ ID NO: 4 - protein, 229 aa, Homo sapiens
MALRRPPRLRLCARLPDFFLLLLFRGCLIGAVNLKSSNRTPVVQEFESVELSCIITDSQTSDPRIEWKKIQDEQTTYVFFDNKIQGDLAGRAEILGKTSLKIWNVTRRDSALYRCEVVARNDRKEIDEIVIELTVQVKPVTPVCRVPKAVPVGKMATLHCQESEGHPRPHYSWYRNDVPLPTDSRANPRFRNSSFHLNSETGTLVRSLLRGVHCCSQGRLWAVLLHCFQ

SEQ ID NO: 5 - protein, 265 aa, Homo sapiens
MALRRPPRLRLCARLPDFFLLLLFRGCLIGAVNLKSSNRTPVVQEFESVELSCIITDSQTSDPRIEWKKIQDEQTTYVFFDNKIQGDLAGRAEILGKTSLKIWNVTRRDSALYRCEVVARNDRKEIDEIVIELTVQVKPVTPVCRVPKAVPVGKMATLHCQESEGHPRPHYSWYRNDVPLPTDSRANPRFRNSSFHLNSETGTLVFTAVHKDDSGQYYCIASNDAGSARCEEQEMEVFTRTQGNQMELTTSALTRRATSDTSHRL

SEQ ID NO: 6 - DNA, 972 bp, Homo sapiens
atggcgaggaggagccgccaccgcctcctcctgctgctgctgcgctacctggtggtcgccctgggctatcataaggcctatgggttttctgccccaaaagaccaacaagtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactggagaatattcctgtgaagcccgcaattctgttggatatcgcaggtgtcctgggaaacgaatgcaagtagatgatctcaacataagtggcatcatagcagccgtagtagttgtggccttagtgatttccgtttgtggccttggtgtatgctatgctcagaggaaaggctacttttcaaaagaaacctccttccagaagagtaattcttcatctaaagccacgacaatgagtgaaaatgtgcagtggctcacgcctgtaatcccagcactttggaaggccgcggcgggcggatcacgaggatttcaagcacacaaaatcctttataatttaaagactccactttag

SEQ ID NO: 7 - DNA, 690 bp, Homo sapiens
atggcgctgaggcggccaccgcgactccggctctgcgctcggctgcctgacttcttcctgctgctgcttttcaggggctgcctgataggggctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttctttccacttaaactctgaaacaggcactttggtaagatctcttctaagaggtgttcactgctgttcacaaggacgactctgggcagtactactgcattgcttccaatga

SEQ ID NO: 8 - DNA, 798 bp, Homo sapiens
atggcgctgaggcggccaccgcgactccggctctgcgctcggctgcctgacttcttcctgctgctgcttttcaggggctgcctgataggggctgtaaatctcaaatccagcaatcgaaccccagtggtacaggaatttgaaagtgtggaactgtcttgcatcattacggattcgcagacaagtgaccccaggatcgagtggaagaaaattcaagatgaacaaaccacatatgtgttttttgacaacaaaattcagggagacttggcgggtcgtgcagaaatactggggaagacatccctgaagatctggaatgtgacacggagagactcagccctttatcgctgtgaggtcgttgctcgaaatgaccgcaaggaaattgatgagattgtgatcgagttaactgtgcaagtgaagccagtgacccctgtctgtagagtgccgaaggctgtaccagtaggcaagatggcaacactgcactgccaggagagtgagggccacccccggcctcactacagctggtatcgcaatgatgtaccactgcccacggattccagagccaatcccagatttcgcaattcttctttccacttaaactctgaaacaggcactttggtgttcactgctgttcacaaggacgactctgggcagtactactgcattgcttccaatgacgcaggctcagccaggtgtgaggagcaggagatggaagtctttacaagaacccagggaaaccagatggagttaactacatccgcactgacgaggagggcgacttcagacacaagtcatcgtttgtga

SEQ ID NO: 9 - DNA, 26 bp, Artificial
  synthetic construct
gggaagatggcgaggaggagccgcca

SEQ ID NO: 10 - DNA, 27 bp, Artificial
  synthetic construct
gtcggacaaagcagagttttataatag

SEQ ID NO: 11 - DNA, 36 bp, Artificial
  synthetic construct
aaccctcgacatggcgctgaggcggccaccgcgact

SEQ ID NO: 12 - DNA, 29 bp, Artificial
  synthetic construct
gtgtctagctctgtccgagtgcatcagct

(19) United States
(12) Patent Application Publication
(10) Pub. No.: US 2006/0079668 A1
(43) Pub. Date: Apr. 13, 2006

(54) Junctional adhesion molecule splice variants

(75) Inventors: Clifford Michael Babbey, Indianapolis, IN (US); Jacquelyn Kaye McEntire, Indianapolis, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company, Indianapolis, IN (US)

(21) Appl. No.: 10/536,366

(22) PCT Filed: Dec. 4, 2003
(86) PCT No.: PCT/US03/37077

§ 371 Date: May 25, 2005

Related U.S. Application Data
(60) Provisional application No. 60/432,702, filed on Dec. 11, 2002.

(51) Int. Cl. A61K 38/17; C07K 14/705; C07H 21/04; C12P 21/06

(52) U.S. Cl. 530/350; 536/23.5; 435/69.1; 435/320.1; 435/325; 514/12

(57) ABSTRACT

This invention provides splice variants of human junctional adhesion molecules (huJAM) and polynucleotides which encode the huJAM splice variants. The invention further provides methods using the molecules of the invention for treating wound healing, cancer, inflammatory disorders, immune system disorders and cardiovascular disorder.

### FIELD OF THE INVENTION

This invention provides splice variants of human junctional adhesion polypeptides huJAM2 and huJAM3, and polynucleotides that encode the splice variants. The invention further provides compositions and methods for using the proteins and polynucleotides of the invention for the treatment and prevention of cancer, cardiovascular disorders, and/or immune system disorders such as autoimmune diseases and inflammatory disorders.

### BACKGROUND OF THE INVENTION

Junctional adhesion molecules (JAM) are members of the immunoglobulin superfamily (IgSf). JAM have two extracellular IgSf domains, a transmembrane segment, and a short cytoplasmic segment. Currently, three distinct JAM proteins, JAM1, JAM2, and JAM3, have been identified in both murine and human sources. JAM are localized to the intercellular boundaries of endothelial and epithelial cells although the tissue distribution pattern for each is distinct (Aurrand-Lions, M. et al. Curr. Top. Microbiol. Immunol. 251:91-98, 2000).

Endothelial cells lining blood vessels form a blood-tissue barrier and such cells are attached to each other by at least two types of complex, junctional structures, adherens junctions (AJ) and tight junctions (TJ), to form a continuous layer of cells. JAM1, JAM2, and JAM3 are concentrated at the sites of cell-cell junctions for both endothelial and epithelial cells and facilitate cell-cell contact through homotypic and/or heterotypic interactions. JAM are also functionally implicated in cell trafficking and cell-fate determination (Malergue, F. et al. Mol. Immunol. 35:1111-1119, 1998).

Leukocytes, it is commonly believed, leave the blood by first adhering to endothelial cells and then migrating through the interendothelial junctions. In doing so they cause disruption of the junctional structures. The process by which leukocytes traverse these junctions is not completely understood, particularly on a molecular level. However, it has been proposed that JAM play a structural role in the control of leukocyte migration across epithelium or endothelium to sites of inflammation. JAM1, localized to TJ, is involved in myeloid cell and neutrophil transmigration (Padura, I., et al. J. Cell Biol. 142:117-127, 1998) while JAM2 may have a role in controlling leukocyte recirculation at secondary lymphoid organs such as lymph nodes and tonsils (Aurrand-Lions, M. et al. Curr. Top. Microbiol. Immunol. 251:91-98, 2000). Much remains to be learned about the role of JAM in leukocyte infiltration and in the inflammation process.

Human JAM (huJAM) is reported to be expressed at high levels in circulating immune cells, both at the mRNA and at the protein level (Williams, L., et al., Mol. Immunology 36:1175-1188, 1999). It is contemplated that a JAM polypeptide on the endothelial cell may interact with a JAM polypeptide on the immune cell thereby inducing transmembrane signaling and the passage of immune cells through the interendothelial junction. It has been reported that JAM2 adheres to T cells through heterotypic interactions with JAM3 and JAM2 must first adhere to JAM3 before it can attach to T-cells (Cunningham, S. et al. J. Biol. Chem. 277:27589-27592, 2002; Arrate, M. et al. J. Biol. Chem. 276:45826-45832, 2001). JAM3 has been shown to be expressed during human cardiogenesis in the structures affected in hypoplastic left heart (Phillips, H. et al. Genomics. 79:475478, 2002).

Numerous publications and databases have reported the full-length, membrane-bound sequence of murine (mu) and human (hu) JAM1, JAM2, and JAM3 polypeptides as well as the polynucleotide sequences encoding the JAM polypeptides (e.g., International Patent Publication Numbers: WO9842739, WO9840483, WO9927098, WO9914241, WO0073452, WO0029583, WO0061623, WO0053758, WO0053749, WO0056754, WO0053758, WO0107459). Polypeptide and polynucleotide sequences encoding extracellular JAM molecules are reported in International Application Number PCT/US02/19800 and International Patent Publication Number WO0053758.

It is well known that the regulated and coordinated expression of adhesion molecules is required for normal vascular function. During inflammation, the cell-cell interactions of the epithelial cell layer are disrupted, resulting in a leaky epithelial barrier, which in turn can lead to various inflammatory and infective disorders. Changes in the adhesion properties of vascular endothelial cells are also observed during tumor growth, wound healing, and angiogenesis. There is great clinical potential and need for variant polypeptide molecules of the full-length, membrane bound JAM, particularly extracellular variants, that can bind JAM ligand and thereby prevent leukocyte transmigration across adherens junctions or tight junctions. Such molecules would be useful for the diagnosis, prevention and treatment of cancer, cardiovascular disorders, and immune system disorders.

### SUMMARY OF THE INVENTION

The present invention addresses the need for huJAM agonists and/or antagonists by providing splice variant forms of huJAM2 and huJAM3 polypeptides, the polynucleotides encoding the polypeptides, and related compositions and methods.

The present invention embodies splice variant huJAM2 (huJAM2sv) and two different splice variant huJAM3 (huJAM3sv1 and huJAM3sv2) polypeptides and their use in treating cancer, cardiovascular disorders, and immune system disorders such as autoimmune diseases and inflammatory disorders.

Amino acid residues 1 to about 29 or 30 of the full-length huJAM proteins (see e.g., SEQ ID NOS: 1-5) encompass a signal peptide that is removed by a signal peptidase enzyme during maturation of the huJAM protein. While huJAM polypeptides can be encoded by a nucleic acid that encodes the signal peptide, this signal peptide is cleaved off and is not present in the mature form of the protein. Alternatively, huJAM polypeptides, including the huJAM2sv, huJAM3sv1 and huJAM3sv2 polypeptides of the present invention, can be encoded by a nucleic acid that lacks sequence encoding the signal peptide.

The invention embodies multiple forms of isolated huJAM2 and huJAM3 splice variant polypeptides including isolated huJAM splice variant polypeptides comprising a polypeptide with a sequence which is at least 99% identical or 100% identical to a sequence selected from the group consisting of: (a) amino acids 1-323 of SEQ ID NO: 2; (b) amino acids 29-323 of SEQ ID NO: 2; (c) amino acids 1-229 of SEQ ID NO: 4; (d) amino acids 31-229 of SEQ ID NO: 4; (e) amino acids 1-265 of SEQ ID NO: 5; (f) amino acids 31-265 of SEQ ID NO: 5; (g) amino acids 1-242 of SEQ ID NO: 5; and (h) amino acids 31-242 of SEQ ID NO: 5. Preferably the isolated huJAM2 and huJAM3 splice variant polypeptides of the invention are capable of binding a huJAM ligand.

One embodiment of the invention embodies isolated and purified nucleic acid molecules including mRNAs, DNAs and cDNAs encoding a polypeptide of the present invention.

The present invention further embodies isolated and purified polynucleotides encoding a huJAM polypeptide comprising a polypeptide with a sequence which is at least 99% identical, or alternatively 100% identical, to a sequence selected from the group consisting of: (a) amino acids 1-323 of SEQ ID NO: 2; (b) amino acids 29-323 of SEQ ID NO: 2; (c) amino acids 1-229 of SEQ ID NO: 4; (d) amino acids 31-229 of SEQ ID NO: 4; (e) amino acids 1-265 of SEQ ID NO: 5; (f) amino acids 31-265 of SEQ ID NO: 5; (g) amino acids 1-242 of SEQ ID NO: 5; and (h) amino acids 31-242 of SEQ ID NO: 5.

Preferably, polynucleotides of the invention have the DNA sequence shown in SEQ ID NOS: 6-8, or the corresponding portion thereof, or variation thereof, that encodes a huJAM polypeptide splice variant comprising a polypeptide with a sequence which is at least 99% identical to, or alternatively 100% identical to, a sequence selected from the group consisting of: (a) amino acids 1-323 of SEQ ID NO: 2; (b) amino acids 29-323 of SEQ ID NO: 2; (c) amino acids 1-229 of SEQ ID NO: 4; (d) amino acids 31-229 of SEQ ID NO: 4; (e) amino acids 1-265 of SEQ ID NO: 5; (f) amino acids 31-265 of SEQ ID NO: 5; (g) amino acids 1-242 of SEQ ID NO: 5; and (h) amino acids 31-242 of SEQ ID NO: 5. Preferably the polynucleotides of the invention encode a polypeptide capable of binding a huJAM ligand.

While SEQ ID NOs: 6, 7 and 8 have nucleic acid sequence encoding the signal peptide, it is contemplated that polynucleotides encoding a huJAM splice variant protein of the invention can lack the nucleic acid sequence encoding the signal peptide and fall within the bounds of the invention.

Additional compositions of the invention are those comprising: (I) a purified, therapeutically effective, extracellular huJAM polypeptide splice variant with an amino acid sequence which is at least 99% identical to, or 100% identical to, a sequence selected from the group consisting of: (a) amino acids 1-323 of SEQ ID NO: 2; (b) amino acids 29-323 of SEQ ID NO: 2; (c) amino acids 1-229 of SEQ ID NO: 4; (d) amino acids 31-229 of SEQ ID NO: 4; (e) amino acids 1-265 of SEQ ID NO: 5; (f) amino acids 31-265 of SEQ ID NO: 5; (g) amino acids 1-242 of SEQ ID NO: 5; and (h) amino acids 31-242 of SEQ ID NO: 5 and (h) and a pharmaceutically acceptable carrier, wherein the carrier is: an aqueous compound including water, saline, and/or buffer; and formulated for oral, rectal, nasal, topical, or parenteral administration. It is contemplated that a composition can comprise one, two, or more different huJAM polypeptides including one or more splice variant forms of a huJAM polypeptide.

The invention further embodies an expression vector comprising a polynucleotide of the invention. The invention further embodies a host cell, e.g., mammalian cells, E. coli, Sf9 cell and yeast cells, transfected with an expression vector of the invention.

The invention also embodies a method for producing a polypeptide of the invention comprising the steps of culturing a host cell of the invention under conditions suitable for expression of a polypeptide of the invention and recovering the polypeptide from the host cell culture medium. The invention further embodies methods of treating an immune system disorder, a cardiovascular disorder, cancer and wound healing comprising administering a therapeutically effective amount of a purified polypeptide of the invention to a mammal in need of such treatment.

### BRIEF DESCRIPTION OF THE FIGURES

FIG. 1 provides an alignment of full-length huJAM2 (SEQ ID NO: 1) and huJAM2sv (SEQ ID NO: 2) polypeptide sequences with putative signal sequence, approximate transmembrane domain and approximate cytoplasmic domain sequences identified. The amino acid residues of huJAM2sv that differ from the corresponding sequence of huJAM2 are in bold type.

FIG. 2 provides an alignment of full-length huJAM3 (SEQ ID NO: 3), huJAM3sv1 (SEQ ID NO: 4), and huJAM3sv2 (SEQ ID NO: 4) polypeptide sequences with putative signal sequence, approximate transmembrane domain and approximate cytoplasmid domain sequences identified. The amino acid residues of huJAM3sv1 and huJAM3sv2 that differ from the corresponding sequence of huJAM3 are in bold type.

FIG. 3 provides the polynucleotide sequence (SEQ ID NO: 6) encoding huJAM2sv (SEQ ID NO: 2).

FIG. 4 provides the polynucleotide sequence (SEQ ID NO: 7) encoding huJAM3sv1 (SEQ ID NO: 4).

FIG. 5 provides the polynucleotide sequence (SEQ ID NO: 8) encoding huJAM3sv2 (SEQ ID NO: 5).

### DETAILED DESCRIPTION OF THE INVENTION

Definitions

The term “isolated” when used in relation to a nucleic acid or protein, means the material is identified and separated from at least one contaminant with which it is ordinarily associated in its natural source. Such a nucleic acid could be part of a vector and/or such nucleic acid or protein could be part of a composition, and still be isolated in that such vector or composition is not part of its natural environment.

As used herein, the term “purified” means the result of any process that removes from a sample a contaminant from the component of interest, such as a protein or nucleic acid. The percent of a purified component is thereby increased in the sample.

The term “homology,” as used herein, refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.”

In the present invention “extracellular huJAM” refers to a form of the said human JAM polypeptide which is essentially free of the signal peptide and the transmembrane and cytoplasmic domains of the full-length huJAM polypeptide (e.g., amino acids from about residue 31 to 229 of SEQ ID NO: 4 for huJAM3sv1 and amino acids from about residue 31 to about residue 242 of SEQ ID NO: 5 for huJAM3sv2). The exact boundaries of where the signal peptide ends and the extracellular domain begins and the exact boundaries of where the extracellular domain ends and the transmembrane domain begins may vary but most likely by no more than about six amino acids at either end of the domain as identified herein. Therefore, an extracellular huJAM signal peptide/extracellular domain boundary as identified in the Examples, Figures, or specification may be shifted in either direction (upstream or downstream) by 6, 5, 4, 3, 2, 1, or 0 amino acids. Additionally, an extracellular huJAM extracellular domain/transmembrane domain boundary as identified in the Examples or specification may be shifted in either direction by 6, 5, 4, 3, 2, 1 or 0 amino acids. All such polypeptides and the nucleic acid molecules encoding them are contemplated by the present invention. For example, extracellular huJAM3sv2 domain is contemplated to extend from about amino acids 31-242 of the full-length huJAM3sv2 polypeptide (SEQ ID NO:5), but it is contemplated that extracellular huJAM3sv2 could span from an amino-terminal amino acid chosen from between amino acids 25 through 37 (inclusive) of the full-length huJAM3sv2 through a carboxy-terminal amino acid from between amino acids 236 through 248 (inclusive) of the full-length huJAM3sv2 as shown in SEQ ID NO: 5.

A polynucleotide or nucleic acid of the present invention can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded regions. A polynucleotide may contain one or more modified nucleotides. “Modified” bases include, for example, tritylated bases and unusual bases such as inosine. A variety of such modifications can be made; thus, “polynucleotide” embraces chemically, enzymatically, or metabolically modified forms.

“Polypeptide variant” as used herein means an “active” polypeptide as defined below, having at least about 98%, more preferably at least 99%, amino acid sequence identity to a reference polypeptide. Polypeptide variants include, for instance, variations of SEQ ID NOs: 2, 4 and 5 wherein one or more amino acid residues are added, substituted or deleted, at the N- or C-terminus or within the sequences, not necessarily contiguously. For example a polypeptide with the sequence shown in SEQ ID NOs: 2, 4 or 5 could be the reference polypeptide and the polypeptide altered from the reference polypeptide would be the polypeptide variant. Ordinarily, a polypeptide variant will have at least about 98% sequence identity, even more preferably at least about 99% amino acid sequence identity with the amino acid sequence described (i.e., the reference polypeptide), with or without the signal peptide.

“Percent (%) amino acid sequence identity” with respect to the amino acid sequences identified herein is defined as the percentage of amino acid residues in a candidate sequence that are identical with the amino acid residues in a reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. For example, the percent identity values used herein can be generated using WU-BLAST-2 [Altschul et al., Methods in Enzymology 266: 460-480 (1996)]. Most of the WU-BLAST-2 search parameters are set to the default values. Those not set to default values, (i.e., the adjustable parameters) are set with the following values: overlap span=1; overlap fraction=0.125; word threshold (T)=11; and scoring matrix=BLOSUM 62. For purposes herein, a percent amino acid sequence identity value is determined by dividing (a) the number of matching identical amino acid residues between the amino acid sequence of the LP polypeptide of interest and the comparison amino acid sequence of interest (i.e., the sequence against which the polypeptide of interest is being compared) as determined by WU-BLAST-2, by (b) the total number of amino acid residues of the polypeptide of interest, respectively.

A “variant polynucleotide” means a nucleic acid molecule encoding an active polypeptide, as defined below, having at least 85% nucleic acid sequence identity with a polynucleotide identified by a SEQ ID NO. of the present invention. Ordinarily, a polynucleotide will have at least 85% nucleic acid sequence identity, more preferably at least 86%, 87%, 88%, 89%, even more preferably at least 90%, 91%, 92%, 93%, 94%, 95%, 96% or 97% nucleic acid sequence identity, yet more preferably at least 98% nucleic acid sequence identity, even more preferably at least 99% nucleic acid sequence identity with the nucleic acid sequence of its corresponding nucleic acid represented by a SEQ ID NO. for the reference polynucleotide (e.g., SEQ ID NO: 6, 7 or 8 of the present invention).

“Percent (%) nucleic acid sequence identity” with respect to the polynucleotide sequences identified herein is defined as the percentage of nucleotides in a candidate sequence that are identical with the nucleotides in the reference sequence after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN, Align-2, Megalign (DNASTAR), or BLAST (e.g., Blast, Blast-2) software. Those slilled in the art can determine appropriate parameters for measuring alignment, including any algorithms needed to achieve maximal alignment over the full length of the sequences being compared. For example, percent nucleic acid identity values can be generated using the WU-BLAST-2 (BlastN module) program (Altschul et al., Methods in Enzymology 266:460-480 (1996)). Most of the WU-BLAST-2 search parameters are set to the default values. Those not set default values (i.e., the adjustable parameters), are set with the following values: overlap span=1; overlap fraction=0.125; word threshold (T)=11; and scoring matrix=BLOSUM62. For purposes herein, a percent nucleic acid sequence identity value is determined by dividing (a) the number of matching identical nucleotides between the nucleic acid sequence of the polypeptide-encoding nucleic acid molecule of interest and the comparison nucleic acid molecule of interest (i.e., the sequence against which the polypeptide-encoding nucleic acid molecule of interest is being compared) as determined by WU-BLAST-2, by (b) the total number of nucleotides of the polypeptide-encoding nucleic acid molecule of interest.

The term “mature protein” or “mature polypeptide” as used herein refers to the form(s) of the protein as would be produced by expression in a mammalian cell. For example, it is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has an SP sequence, as well as the cleavage point for that sequence, are known in the art. A cleavage point may exist within the N-terminal domain between amino acid 10 and amino acid 35. More specifically the cleavage point is likely to exist after amino acid 15 but before amino acid 31. As one of ordinary skill would appreciate, however, cleavage sites sometimes vary from organism to organism and may even vary from molecule to molecule within a cell and cannot be predicted with absolute certainty. Optimally, cleavage sites for a secreted protein are determined experimentally by amino-terminal sequencing of the one or more species of mature proteins found within a purified preparation of the protein.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes naturally occurring proteogenic L-amino acids; D-amino acids; chemically modified amino acids, such as amino acid analogs and derivatives; naturally occurring non-proteogenic amino acids such as norleucine, β-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The terms “treating”, “treatment” and “therapy” as used herein refer to curative therapy, prophylactic therapy, and preventive therapy. An example of “preventive therapy” is the prevention (or lessened likelihood) of a targeted pathological condition or disorder. Those in need of treatment include those already with the disorder as well as those prone to have the disorder or those in whom the disorder is to be prevented.

The term “agonist” when used with reference to a huJAM refers to a molecule which intensifies or mimics the biological activity of said huJAM. Agonists may include proteins, nucleic acids, carbohydrates, small molecules, or any other compound or composition which modulates the activity of huJAM either by directly interacting with huJAM or by acting on component(s) of the biological pathway in which huJAM participates.

The term “antagonist” when used with reference to a huJAM refers to a molecule which inhibits or attenuates the biological activity of said huJAM. Antagonists may include proteins, antibodies, nucleic acids, carbohydrates, small molecules, or any other compound or composition which modulates the activity of huJAM either by directly interacting with huJAM or by acting on component(s) of the biological pathway in which huJAM participates.

A “therapeutically-effective amount” is the minimal amount of active agent (e.g., a splice variant huJAM polypeptide) necessary to impart therapeutic benefit to a mammal. For example, a “therapeutically-effective amount” to a mammal is such an amount that induces, ameliorates or otherwise causes an improvement in the pathological symptoms, disease progression, physiological conditions associated with or resistance to succumbing to the aforedescribed disorder.

“Carriers” as used herein include pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol, and PLURONIC™.

“Active” or “activity” in the context of variants of the polypeptides of the invention refers to retention of a biologic function of the unmodified polypeptide (e.g., huJAM2sv, huJAM3sv1, huJAM3sv2) and/or the ability to bind to a receptor or ligand much as would an unmodified polypeptide of the invention, and/or the ability to induce production of an antibody against an antigenic epitope possessed by the polypeptide at levels near that of the unmodified polypeptide. More specifically, “biological activity” refers to a biological function (either inhibitory or stimulatory) caused by a reference polypeptide. Exemplary biological activities include, but are not limited to, the ability of such molecules to induce or inhibit infiltration of inflammatory cells (e.g., leukocytes) into a tissue, to induce or inhibit adherence of a leukocyte to an endothelial or epithelial cell, to stimulate or inhibit T-cell proliferation or activation, to stimulate or inhibit cytokine release by cells or to increase or decrease vascular permeability.

Compositions and Methods of the Invention

The present invention is based in part upon the discovery and synthesis of huJAM2 and huJAM3 splice variant proteins (huJAM2sv (SEQ ID NO: 2), huJAM3sv1 (SEQ ID NO: 4) and huJAM3sv2 (SEQ ID NO: 3)) and their use in treating, preventing, and diagnosing cancer, cardiovascular disorders, and immune system disorders such as autoimmune diseases and inflammatory disorders. HuJAM2sv contains a 61 bp exon that is not found in huJAM2.

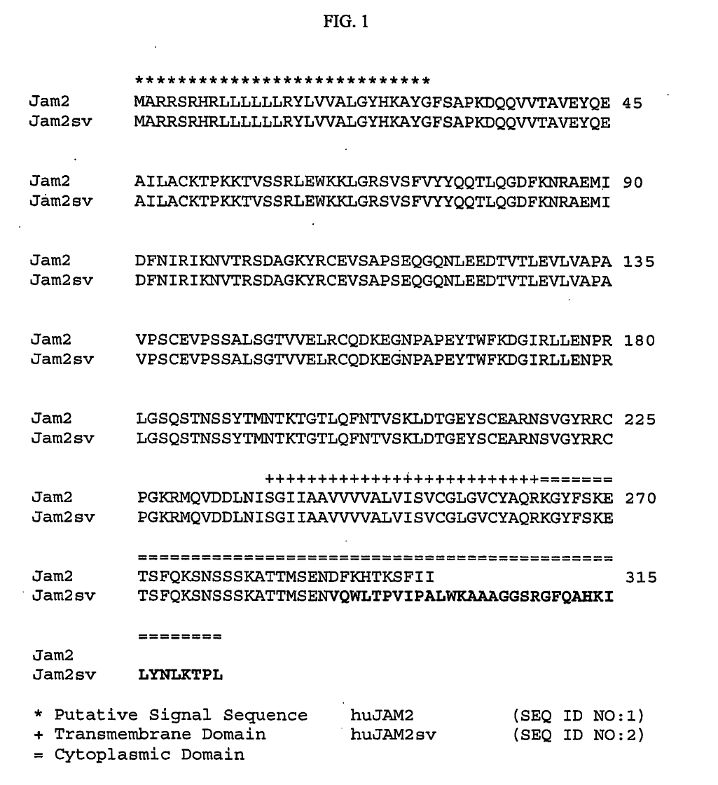

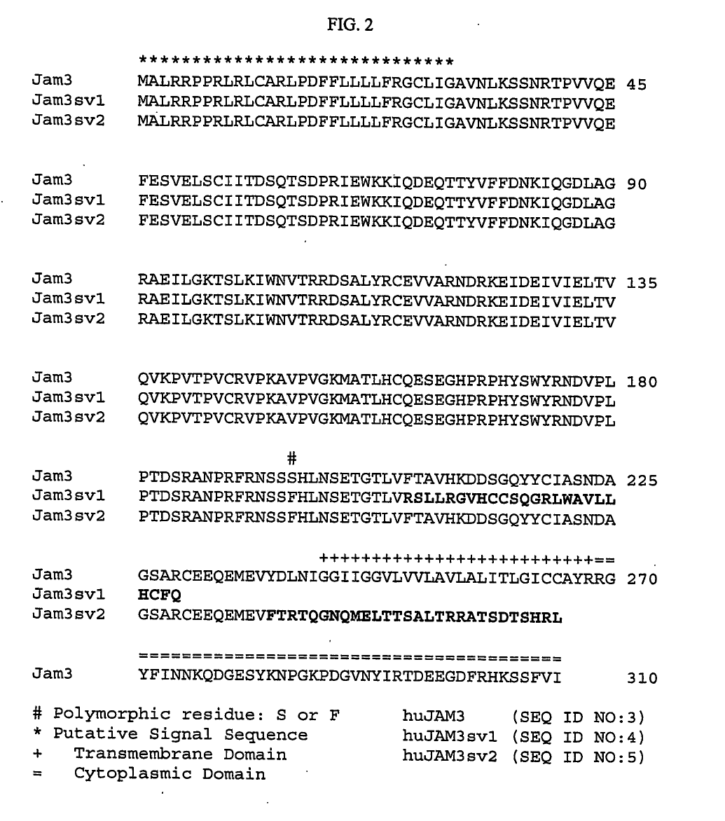

The additional exon is located between exons 9 and 10 of JAM2. The inclusion of this exon causes a frame shift, resulting in a longer open reading frame when compared to huJAM2 (see FIG. 1). The frameshift also results in the loss of a PDZ binding site. Since this site is required for interaction with AF-6 and ZO-1, two cytoplasmic proteins shown to bind huJAM2 and which are important in the formation/maintenance of tight junctions, this loss could be significant in the regulation of intercellular junctions. In particular, the loss of the pDZ domain indicates that LP2012 may not be anchored to the cytoplasmic junctional scaffold. This increased motility may give huJAM2sv a role in escorting bound T-cells across the endothelia. HuJAM3sv1 is a splice variant of huJAM3 that uses an alternative splicing site at the 3′ end of exon 5, resulting in a 19 bp insertion not found in huJAM3. The aleternative splicing of huJAM3sv1 causes a frame shift, resulting in a different carboxy terminus sequence and a short stop comparted to huJAM3. As a consequence of these changes, the transmembrane domain found in huJAM3 is not present in huJAM3sv1 (see FIG. 2). HuJAM3sv2 is a splice variant of huJAM3 that is missing exon 7 of huJAM3. The alternative splicing of huJAM3sv2 causes a frame shift in the open reading frame, resulting in a different carboxy terminal sequence and a short stop compared to huJAM3. As a consequence of these changes, the transmembrane domain found in huJAM3 is not present in huJAM3sv2. in comparison to huJAM3sv1, huJAM3sv2 contains the entire second Ig domain, whereas this domain in huJAM3sv1 is truncated.

Identification and Preparation of Splice Variant HuJAM Polypeptides

Nucleic acid encoding a huJAM splice variant polypeptide may be obtained from a cDNA library prepared from tissue believed to possess the huJAM mRNA and to express it at a detectable level. Libraries can be screened with probes (such as antibodies to a huJAM polypeptide or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual, Cold Spring Harbor Laboratory Press, NY (1989). An alternative means to isolate the gene encoding LP polypeptide is to use PCR methodology [Sambrook et al., supra; Dieffenbach et al., PCR Primer: A Laboratory Manual, Cold Spring Harbor Laboratory Press, NY (1995)]. Further details of the cloning and expression of the polypeptides of the invention are in the Examples 1 and 2 herein.

The polypeptides of the present invention are human junction adhesion molecules that are splice variants of the full length molecules. The polypeptides may have a signal peptide sequence to enable protein transport within the cell; however, this signal peptide sequence is not present in the mature huJAM2sv1, huJAM3sv1 or huJAM3sv2 polypeptides as they exist when outside the cell.

A signal peptide, comprised of about 10-30 hydrophobic amino acids, targets the nascent protein from the ribosome to the endoplasmic reticulum (ER). Once localized to the ER, the proteins can be further directed to the Golgi apparatus within the cell. The Golgi distributes proteins to vesicles, lysosomes, the cell membrane, and other organelles. Proteins targeted to the ER by a signal sequence can be released from the cell into the extracellular space. For example, vesicles containing proteins to be moved outside the cell can fuse with the cell membrane and release their contents into the extracellular space via a process called exocytosis. Exocytosis can occur constitutively or after receipt of a triggering signal. In the latter case, the proteins are stored in secretory vesicles until exocytosis is triggered. Proteins that transit through this pathway are either released into the extracellular space or retained in the plasma membrane. Protein that are retained in the plasma membrane (e.g., full-length huJAM), contain one or more transmembrane domains, each comprised predominantly of hydrophobic amino acid residues.

The common structure of signal peptides from various proteins is typically described as a positively charged n-region, followed by a hydrophobic h-region and a neutral but polar c-region. The (−3, −1) rule states that the residues at positions −3 and −1 (relative to the signal peptide cleavage site) must be small and neutral for cleavage to occur correctly. In many instances the amino acids comprising the signal peptide are cleaved off the protein during transport or once its final destination has been reached. Specialized enzymes, termed signal peptidases, are responsible for the removal of the signal peptide sequences from proteins. These enzymes are activated once the signal peptide has directed the protein to the desired location.

Polypeptides of the invention may be produced recombinantly, not only directly, but also as a fusion polypeptide with a heterologous polypeptide, which optionally may be a signal sequence, or other polypeptide having a specific cleavage site at the N-terminus of the mature protein or polypeptide. In general, the signal sequence may be a component of an expression vector, or it may be a part of the polypeptide-encoding DNA that is inserted into such a vector. For E. coli expression, the signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader (including Saccharomyces and Kluyveromyces cc-factor leaders, the latter described in U.S. Pat. No. 5,010,182), or acid phosphatase leader, the C. albicans glucoamylase leader (EP 362,179), or the signal described in WO 90/13646. In mammalian cell expression, mammalian signal sequences may be used to direct secretion of the protein, such as signal sequences from secreted polypeptides of the same or related species as well as viral secretory leaders.

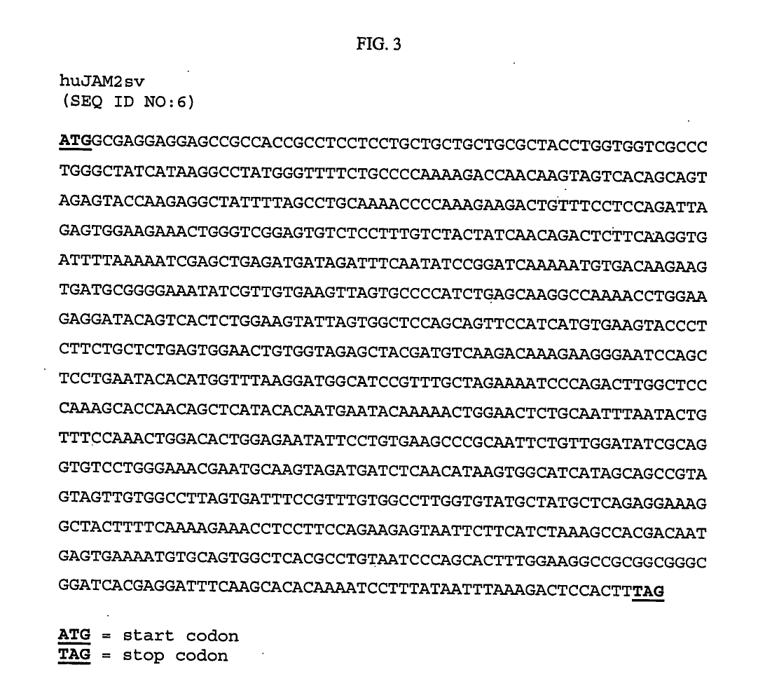

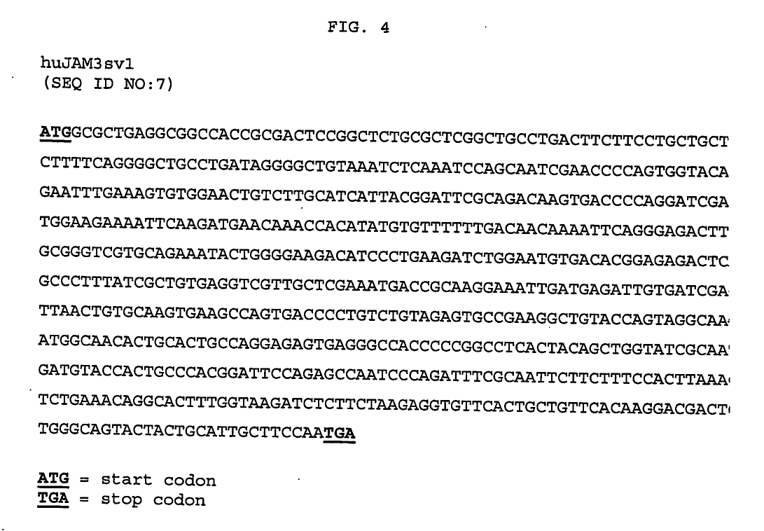

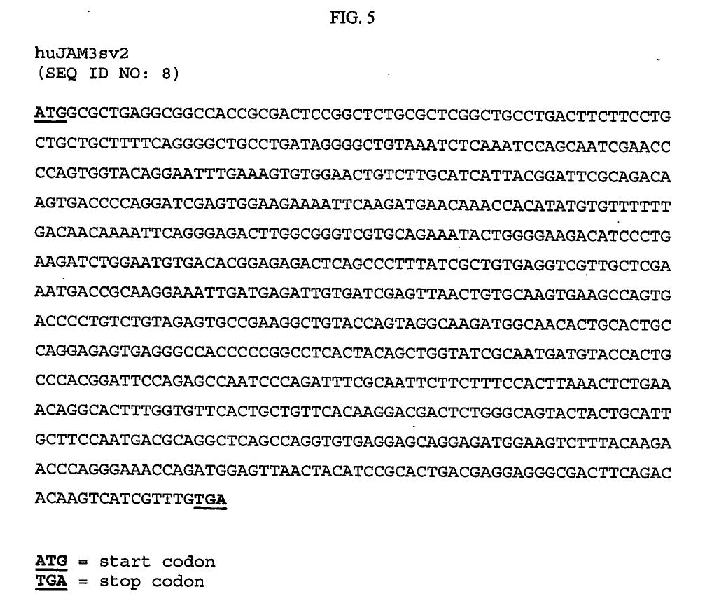

Also provided by the present invention are nucleic acid compositions encoding the polypeptides of the invention. These nucleic acids are shown in SEQ ID NOS: 6-8 (FIGS. 3 through 5 respectively).

Splice Variant HuJAM Polypeptide and Polynucleotide Variants

The present invention encompasses variants of the polynucleotide sequence disclosed in SEQ ID NOS: 6, 7 and 8 and their complementary strands. These polynucleotides encode a huJAM splice variant polypeptide, either full length or mature form, with or without a transmembrane domain. Especially preferred are polynucleotide variants containing alterations, which produce silent substitutions (i.e., no change in amino acid encoded thereby), additions, or deletions, but do not alter the properties or activities of the encoded polypeptide. Nucleotide variants produced by silent substitutions due to the degeneracy of the genetic code are preferred.

The term “variant” refers to a polynucleotide or polypeptide differing from a polynucleotide sequence or polypeptide of the present invention, but retaining essential properties thereof. Generally, variants are closely similar overall in structural and sequence identity, and, in many regions, identical to a polynucleotide or polypeptide of the present invention. The term “variant” is further described in the definitions herein.

The present invention also encompasses variants of the huJAM polypeptide sequences disclosed in SEQ ID NOS: 2, 4 and 5. The present invention is directed to polypeptides that comprise an amino acid sequence that is at least 98%, more preferably at least 99% identical or 100% identical to a polypeptide with a sequence shown in SEQ ID NOs: 2, 4 or 5, either the full length or mature form of the polypeptide; additionally, for SEQ ID NO: 5, either with or without the transmembrane region. Alterations in amino residues of a polypeptide sequence may occur, e.g., at the amino or carboxy terminal positions or anywhere between these terminal positions, interspersed either individually among residues in the sequence or in one or more contiguous sections, portions, or fragments within the sequence.

Variants may be produced by mutagenesis techniques or by direct synthesis using known methods of protein engineering and recombinant DNA technology. Such variants may be generated to improve or alter the characteristics of a polypeptide or may occur unintentionally. For instance, one or more amino acids can often be deleted from the N-terminus or C-terminus of a secreted polypeptide without a substantial loss of biological function. Alternatively, variants may be found to exist naturally in a percentage of the population and be cloned directly therefrom.

Ample evidence demonstrates that polypeptide or polynucleotide variants can retain a biological activity similar to that of the naturally occurring protein. Moreover, even if deleting one or more amino acids from the N-terminus or C-terminus of the polypeptide results in modification or loss of one or more biological functions, other biological activities may be retained.

It is preferable that polypeptide variants of the present invention retain a biological activity of the reference huJAM molecule such as, e.g., ligand binding or antigenicity. Such variants include, e.g., deletions, insertions, inversions, repeats, and substitutions selected so as to have little effect on activity using general rules known in the art. For example, teachings on making phenotypically silent amino acid substitutions are provided, e.g., by Bowie, et al. (1990) Science 247:1306-1310. One technique compares amino acid sequences in different species to identify the positions of conserved amino acid residues since changes in an amino acid at these positions are more likely to affect a protein function. In contrast, the positions of residues where substitutions are more frequent generally indicates that amino acid residues at these positions are less critical for a protein function. Thus positions tolerating amino acid substitutions typically may be modified while still maintaining a biological activity of a protein.

Modifications of Splice Variant HuJAM Polypeptides

Polypeptides of the invention are composed of amino acids joined to each other by peptide bonds or modified peptide bonds, i.e., peptide isosteres, and may contain amino acids other than the gene-encoded amino acids. The polypeptides may be modified by either natural processes, such as posttranslational processing, or by chemical modification techniques which are well known in the art. Such modifications are well-described in the art. Modifications can occur anywhere in the polypeptides, including the peptide backbone, the amino acid side-chains and the amino or carboxyl termini. The same type of modification may be present in the same or varying degrees at several sites in a given polypeptide. Also, a given polypeptide may contain many types of modifications. The polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, Creighton, Proteins—Structure and Molecular Properties, 2nd Ed., W. H. Freeman and Company, New York (1993); Johnson, Posttransational Covalent Modification of proteins, Academic Press, New York, pp. 1-12 (1983); Seifter et al., Meth. Enzymol. 182: 62646 (1990); Rattan et al., Ann. NY Acad. Sci. 663: 48-62 (1992).

Polypeptides of the present invention may also be modified to form fusion molecules comprising a polypeptide of the invention fused to a heterologous polypeptide. In one embodiment, such a fusion molecule comprises a fusion of a polypeptide of the invention with a tag polypeptide which provides an epitope to which an anti-tag antibody can selectively bind. The epitope tag is generally placed at the amino- or carboxyl-terminus of the polypeptide of the invention. The presence of such epitope-tagged forms of polypeptides can be detected using an antibody against the tag polypeptide. Also, provision of the epitope tag enables a polypeptide to be readily purified by affinity purification using an anti-tag antibody or another type of affinity matrix that binds to the epitope tag. In an alternative embodiment, the fusion molecule may comprise a fusion of a polypeptide of the invention with an immunoglobulin or a particular region of an immunoglobulin. For a bivalent form of the fusion molecule, such a fusion could be to the Fc region of an IgG molecule.

Expression of huJAM Polypeptides

Recombinant expression vectors are typically self-replicating DNA or RNA constructs containing a desired gene to be expressed operably linked to a promoter and optionally other control elements recognized in a suitable host cell. The specific type of control elements necessary to effect expression depends on the host cell used and the level of expression desired. Proteins can be expressed in mammalian cells (e.g., CHO, NSO, COS cells), yeast, bacteria, or other cells under the control of appropriate promoters.

Vectors, as used herein, encompass plasmids, viruses, bacteriophage, integratable DNA fragments, and other vehicles that enable the integration of DNA fragments into the genome of the host although, optionally, expression can occur transiently without integration. Plasmids are the most commonly used form of vector, but many other forms of vectors that perform an equivalent function are also suitable for use (see, e.g. Pouwels, et al. (1985 and Supplements) Cloning Vectors: A Laboratory Manual Elsevier, N.Y.; and Rodriquez, et al. (eds.) (1988) Vectors: A Survey of Molecular Cloning Vectors and Their Uses Buttersworth, Boston, Mass.).

Both expression vectors and cloning vectors contain a nucleic acid sequence that enables the vector to replicate in one or more selected host cells. Such sequences are well known for a variety of bacteria, yeast, and viruses. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement autotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the polypeptide-encoding nucleic acid, such as DHFR or thymidine kinase. An appropriate host cell when wild-type DHFR is employed is the CHO cell line deficient in DHFR activity, prepared and propagated as described Urlaub and Chasin, Proc. Natl. Acad. Sci. USA 77:4216 (1980). A suitable selection gene for use in yeast is the trpl gene present in the yeast plasmid YRp7 [Stinchcomb et al., Nature 282:39 (1979); Kingsman et al., Gene 7:141-52 (1979); Tschumper et al., Gene 10:157-66 (1980)]. The trpl gene provides a selection marker for a mutant strain of yeast lacking the ability to grow in tryptophan, for example, ATCC No. 44076 or PEPC1 [Jones, Genetics 85: 23 (1977)].

Expression vectors contain a promoter operably linked to the polypeptide-encoding nucleic acid sequence to direct mRNA synthesis. Promoters recognized by a variety of potential host cells are well known. Promoters for use in bacterial systems also will contain a Shine-Dalgarno sequence operably linked to the DNA encoding a polypeptide of interest.

Transcription of a DNA encoding a polypeptide by higher eukaryotes may be increased by inserting an enhancer sequence into the vector. Enhancers are cis-acting elements of DNA, usually about from 10 to 300 bp, that act on a promoter to increase its transcription level. Many enhancer sequences are now known from mammalian genes (globin, elastase, albumin, a-ketoprotein, and insulin). Typically, however, one will use an enhancer from a eukaryotic-cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the LP polypeptide coding sequence.

Expression vectors used in eukaryotic host cells will also contain sequences necessary for the termination of transcription and optionally for stabilizing the mRNA. Such sequences are commonly available from the 5′ and occasionally 3′ untranslated regions of eukaryotic or viral DNAs or cDNAs. These regions contain nucleotide segments transcribed as polyadenylated fragments in the untranslated portion of the mRNA encoding an LP polypeptide.

The description below relates primarily to production of polypeptides of the invention by culturing cells transformed or transfected with a vector containing a polypeptide-encoding nucleic acid. It is, of course, contemplated that alternative methods, which are well known in the art, may be employed to prepare the polypeptides. For instance, the polypeptide sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc. 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of a polypeptide of the invention may be chemically synthesized separately and combined using chemical or enzymatic methods to produce a full-length polypeptide.

Host cells are transfected or transformed with expression vectors or cloning vectors described herein for polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra. Methods of transfection are known to the ordinarily skilled artisan, for example, CaPO₄ and electroporation.

Suitable host cells for cloning or expressing the nucleic acid (e.g., DNA) in the vectors herein include prokaryote, yeast, or higher eukaryote cells. Suitable prokaryotes include but are not limited to E. coli K12 strain MM294 (ATCC 3 1.446); E. coli X1 776 (ATCC 3 1.537); E. coli strain W3110 (ATCC 27.325) and K5 772 (ATCC 53.635). Other suitable prokaryotic host cells include Enterobacter, Erwinia, Klebisella, Proteus, Salmonella, Serratia, and Shigeila, as well as Bacilli, Pseudomona, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. Alternatively, in vivo methods of cloning, e.g., PCR or other nucleic acid polymerase reactions, are suitable.

In addition to prokaryotes, eukaryotic microbes such as filamentous fungi or yeast are suitable cloning or expression hosts for polypeptide expressing vectors. Saccharomyces cerevisiae is a commonly used lower eukaryotic host microorganism. Many others are used by those in the art.

Suitable host cells for the expression of glycosylated or nonglycosylated polypeptides of the invention are derived from multicellular organisms. Examples of invertebrate cells include insect cells such as Drosophila S2 and Spodoptera Sp, Spodoptera high5 as well as plant cells. Examples of useful mammalian host cell lines include Chinese hamster ovary (CHO) and COS cells. Additional examples include the monkey kidney CV1 line transformed by SV40 (COS-7, ATCC CRL 1651); human embryonic kidney line [293 or 293 cells subcloned for growth in suspension culture, Graham et al., J. Gen Virol. 36:59-74 (1977)]; Chinese hamster ovary cells/−DHFR [CHO, Urlaub and Chasin, Proc. Natl. Acad. Sci. USA. 77:4216-20 (1980)]; mouse sertoli cells [TM4, Mather, Biol. Reprod. 23:243-52 (1980)]; human lung cells (W138. ATCC CCL 75); human liver cells (Hep G2, HB 8065); and mouse mammary tumor (MMT 060562, ATCC CCL51). The selection of the appropriate host cell is within the skill in the art.

Polypeptide Purification

Gene amplification and/or expression may be measured in a sample directly, for example, by conventional Southern blotting, Northern blotting to quantitate the transcription of mRNA [Thomas, Proc. Natl. Acad. Sci. USA 77:5201 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal and may be prepared in any mammal. Conveniently, the antibodies may be prepared against a native sequence provided herein or against exogenous sequence fused to a polypeptide-encoding DNA and encoding a specific antibody epitope.

Forms of polypeptides of the invention may be recovered from culture medium or from host cell lysates. Cells employed in expression of polypeptides of the invention can be disrupted by various physical or chemical means, such as freeze-thaw cycling, sonication, mechanical disruption, or cell lysing agents.

It may be desirable to purify the polypeptides away from another recombinant cell polypeptide. The following procedures are exemplary of suitable purification procedures: fractionation on an ion-exchange column; ethanol precipitation; reversed-phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of polypeptides. Various methods of protein purification may be employed and such methods are known in the art and described, for example, in Deutscher, Methods in Enzymology 182:83 (1990) and Scopes, Protein Purification: Principles and Practice, Springer-Verlag, NY (1982). The purification step(s) selected will depend, for example, on the nature of the production process used and the particular polypeptide produced.

Polypeptide Analysis

Many types of analyses can be performed with the polypeptides of the present invention to demonstrate their role in the development, pathogenesis, and treatment of cancer, cardiovascular disease and immune related disease, e.g., inflammation. Certain analyses are exemplified in the Examples herein. Additional protocols for the analyses may be found in the art.

The location of tissues expressing the polypeptides of the invention can be identified by determining mRNA expression in various human tissues. Such a measurement can be made, for example, by Northern blotting, dot blotting, or in situ hybridization based on the sequences provided herein. Alternatively, antibodies may be used that recognize specific duplexes. The location of a gene in a specific tissue can be performed, for example, by Southern blotting.

Cell-based assays using a cell type (optionally known to be involved in a particular disease) are transfected with a vector expressing a polypeptide of the invention. Such cells are monitored for phenotypic changes, for example T-cell proliferation by mixed lymphocyte reaction, inflammatory cell infiltration, cytokine levels, JAM expression level variation, ligand binding and reaction to particular antibodies. While transiently-transfected cells can be used, stable cell lines expressing extracellular huJAM are preferred.

Animal models can be used to further understand the role of the polypeptides of the invention as demonstrated. Polypeptides of the invention may be used for blocking homotypic and/or heterotypic JAM signaling or JAM interactions and therefore may be useful for the prevention, treatment and diagnosis of cardiovascular disease.

Pharmaceutical Compositions

When the coding sequence for a polypeptide encodes a protein which binds to another protein as is the case for splice variant huJAM polypeptides of the present invention, the polypeptide can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor polypeptide can be used to isolate correlative ligand(s). Screening assays can be designed to find lead compounds that mimic the biological activity of a native polypeptide of the invention or a receptor for an polypeptide of the invention. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays.

Methods of Treatment

HuJAM2sv1, huJAM3sv1 and huJAM3sv2 (full length or mature, with or without a transmembrane domain, and variants thereof) are useful for the prevention, diagnosis, and treatment of cancer, cardiovascular disorders and immune system disorders such as autoimmune diseases and inflammatory disorders.

Particular cancers suitable for treatment with the polypeptides of the invention include, but are not limited to, acute myelogenous leukemias including acute monocytic leukemia, acute myeloblastic leukemia, acute promyelocytic leukemia, acute myelomonocytic leukemia, acute erythroleukemia, acute megakaryocyticleukemia, and acute undifferentiated leukemia, etc.; and chronic myelogenous leukemias including chronic myelomonocytic leukemia and chronic granulocyticleukemia. Additional cancers suitable for treatment with the polypeptides of the invention inculde, but are not limited to, adenocarcinoma, lymphoma, melanoma, myeloma, Hamartoma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

Particular cardiovascular disorder suitable for treatment with the polypeptides of the invention include, but are not limited to, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery.

Particular immune system disorders suitable for treatment with the polypeptides of the invention include, but are not limited to, inflammatory disorders, acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, sepsis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma.

Therapeutic formulations are prepared for storage by mixing the active ingredient having the desired degree of purity with optional pharmaceutically acceptable carriers, excipients or stabilizers [Remington's Pharmaceutical Sciences 16th edition (1980)], in the form of lyophilized formulations or aqueous solutions.

The formulation herein may also contain more than one active compound as necessary for the particular indication being treated, preferably those with complementary activities that do not adversely affect each other. Such molecules are suitably present in combination in amounts that are effective for the purpose intended.

The formulations to be used for in vivo administration must be sterile. This is readily accomplished by filtration through sterile filtration membranes.

The active agents of the present invention are administered to a mammal, preferably a human, in accord with known methods, such as intravenous administration as a bolus or by continuous infusion over a period of time, by intramuscular, intraperitoneal, intracerebral, intracerobrospinal, subcutaneous, intra-articular, intrasynovial, intrathecal, intraoccular, intralesional, oral, topical, inhalation, pulmonary, and/or through sustained release.

For the prevention or treatment of disease, the appropriate dosage of an active agent or the effective dose, will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective does for human therapy. Interspecies scaling of effective doses can be performed following the principles laid down by Mordenti and Chappell, “The Use of Interspecies Scaling in Toxicokinetics,” in Toxicokinetics and New Drug Development, Yacobi et al., Eds., Pergamon Press, NY 1989, pp. 4246.

Guidance as to particular dosages and methods of delivery is provided in the literature; see, for example, U.S. Pat. Nos. 4,657,760, 5,206,344 or 5,225,212. It is within the scope of the invention that different formulations will be effective for different treatment compounds and different disorders, that administration targeting one organ or tissue, for example, may necessitate delivery in a manner different from that to another organ or tissue. Moreover, dosages may be administered by one or more separate administrations or by continuous infusion. For repeated administrations over several days or longer, depending on the condition, the treatment is sustained until a desired suppression of disease symptoms occurs. However, other dosage regimens may be useful. The progress of this therapy is readily monitored by conventional techniques and assays.

Having generally described the invention, the same will be more readily understood by reference to the following examples, which are provided by way of illustration and not by way of limitation.

### EXAMPLES

### Example 1

### Identification and Cloning of HuJAM2sv1

The splice variant of human JAM2 identified by the applicants (HuJAM2sv1) was discovered using the polymerase chain reaction (PCR) method. Oligonucleotide primers were designed to hybridize to the 5′ and 3′ untranslated region of HuJAM2. Although many various primers may be used, the primers used in the PCR reaction had the following sequences:
5′-GGGAAGATGGCGAGGAGGAGCCGCCA-3′ (SEQ ID NO: 9) and
5′-GTCGGACAAAGCAGAGTTTTATAATAG-3′ (SEQ ID NO: 10).
   The 50 μl PCR reaction contained 1×Pfu buffer (Stratagene, LaJolla, Calif.), 0.2 mM dNTPs, 20 picomoles of each primer, 1.25 units Pfu DNA polymerase (Stratagene) and 1 ng human heart cDNA (Clontech, Palo Alto, Calif.). The PCR reaction was cycled through about 40 standard amplification cycles. The success of the amplification was confirmed by viewing 10 μl of the resulting PCR reaction on a 1.2% E-gel (Invitrogen, Carlsbad, Calif.). The remaining PCR product was concentrated to 25 μl using a Microcon YM-50 column (Millipore, Bedford, Mass.) according to manufacturer's instructions.

Overhang A nucleotide residues were added to the purified blunt-ended PCR product following standard procedures. The sample was then purified using a PCR cleanup kit (Qiagen, Valencia, Calif.). The PCR product was then cloned into a pCRII TOPO vector (Invitrogen) according to manufacturer's protocol and transformed into OneShot cells (Invitrogen). Full-length HuJAM2 sequence was obtained for 91 resulting clones and the sequence was compared to that of HuJAM2 wild type sequence. Clones differing from the published sequence were aligned with the genomic sequence to determine if they were splice variants based on the presence of conserved 5′ GT and 3′ AG sequences found in introns. The clone determined to be a splice variant was analyzed for novelty using a BLAST program. One clone from the 91 clones sequenced was determined to be a splice variant of HuJAM2. This clone was named HuJAM2sv1; its polynucleotide sequence is shown herein in SEQ ID NO: 6 and the polypeptide sequence encoded by this polynucleotide is shown herein in SEQ ID NO: 2.

### Example 2

### Identification and Cloning of HuJAM3sv1 and HuJAM3sv2

The splice variants of human JAM3 identified by the applicants (HuJAM3sv1 and HuJAM3sv2) were discovered using the polymerase chain reaction (PCR) method as described in Example 1 above with the exception that the source of DNA was human testis cDNA (Clontech). Oligonucleotide primers were designed to hybridize to the 5′ and 3′ untranslated region of HuJAM3. Although many various primers may be used, the primers used in the PCR reaction had the following sequences:
5′-AACCCTCGACATGGCGCTGAGGCGGCCACCGCGACT-3′ (SEQ ID NO: 11) and
5′-GTGTCTAGCTCTGTCCGAGTGCATCAGCT-3′ (SEQ ID NO: 12). The amplified DNA was prepared and analyzed as described in Example 1.

Sequence data was obtained for 91 clones. The sequences were compared to that of wild-type huJAM3. Clones differing from the published sequence were aligned with genomic sequence to determine if they were splice variants (based on the conserved 5′ GT and 3′ AG sequences found in the introns) or artifacts. The clones determined to be splice variants were analyzed for novelty by using the BLAST program.

The sequence described for huJAM3sv1 was found twice in the 91 clones sequenced. The huJAM3sv1 polynucleotide sequence is that shown in SEQ ID NO: 7 and the polypeptide encoded by this polynucleotide sequence is that shown in SEQ ID NO: 4. The huJAM3sv2 sequence was found once in the 91 clones. The huJAM3sv2 polynucleotide sequence is that shown in SEQ ID NO: 8 and the polypeptide encoded by this polynucleotide sequence is that shown in SEQ ID NO: 5.

### Example 3 Expression and Purification in E. coli

The bacterial expression vector pQE60 is used for bacterial expression in this example although other bacterial expression vectors are commercially available (QIAGEN, Inc., Chatsworth, Calif.). pQE60 encodes an ampicillin antibiotic resistance gene (Amp) and contains a bacterial origin of replication (ori), an IPTG-inducible promoter, a ribosome binding site (RBS), six codons encoding histidine (His6 tag) residues that allow affinity purification using nickel-nitrilo-tri-acetic acid (Ni-NTA) affinity resin sold by QIAGEN, Inc., and single restriction enzyme cleavage sites suitable for cloning (i.e., in a multiple cloning site). These elements are arranged such that a DNA fragment encoding a polypeptide of interest can be operably linked in such a way as to produce that polypeptide with the six His residues covalently linked to the carboxyl terminus of that polypeptide. However, a polypeptide coding sequence can optionally be inserted in such a way that translation of the six His codons is prevented and, therefore, a polypeptide is produced with no 6× His tag.

The nucleic acid sequence encoding the desired portion of huJAM2sv1, huJAM3sv1 and huJAM3sv2 lacking the hydrophobic leader sequence is amplified from a clone using PCR oligonucleotide primers, which anneal, one upstream (or 5′) and one downstream (or 3′), to the desired portion of the polypeptide. Exemplary primers for may hybridize to vector sequence adjacent to the JAM sequence in the clones described in Examples 1 and 2 above. Additional nucleotides containing restriction sites to facilitate cloning in the pQE60 vector may be added to the 5′ and 3′ sequences, respectively.

The amplified nucleic acid fragments and the vector pQE60 are digested with appropriate restriction enzymes and the digested DNAs are then ligated together. Insertion of the polypeptide DNA into the restricted pQE60 vector places the polypeptide coding region including its associated stop codon downstream from the IPTG-inducible promoter and operably linked in-frame with an initiating AUG codon. The associated stop codon prevents translation of the six His codons downstream of the insertion point.

The ligation mixture is transformed into E. coli cells using standard procedures. E. coli strain M15/rep4, containing multiple copies of the plasmid pREP4, which expresses the lac repressor and confers kanamycin resistance (Kan^{r}), may be used in carrying out the example described herein. This strain, which is only one of many that are suitable for expressing polypeptides, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Bacteria containing the desired cloned constructs are grown overnight in liquid culture in LB media (Sigma Corp. St. Louis, Mo.) supplemented with both ampicillin (100 μg/ml) and kanamycin (25 μg/ml). The culture is used to inoculate a large culture, at a dilution of approximately 1:25 to 1:250. The cells are grown to an optical density at 600 nm of between 0.4 and 0.6. Isopropyl-b-D-thiogalactopyranoside (IPTG) is added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for 3-4 hours at 4° C. in 6 M guanidine-HCl, pH 8.0. The cell debris is removed by centrifugation, and the supernatant containing the polypeptide is dialyzed against 50 mM Na-acetate buffer pH 6.0, supplemented with 200 mM NaCl. Alternatively, a polypeptide can be successfully refolded by dialyzing it against 500 mM NaCl, 20% glycerol, 25 mM Tris/HCl pH 7.4, containing protease inhibitors.

If insoluble protein is generated, the protein is made soluble according to known method steps. After renaturation, the polypeptide is purified by ion exchange, hydrophobic interaction, and size exclusion chromatography. Alternatively, an affinity chromatography step such as an antibody column is used to obtain purified polypeptide. The purified polypeptide may be concentrated and is stored at 4° C. or frozen at −40° C. to −120° C.

### Example 4

### Expression in Mammalian Cells

A typical mammalian expression vector contains at least one promoter element, the polypeptide coding sequence, and signal sequence required for the termination of transcription and polyadenylation of the transcript. Additional optional elements include enhancer element, a Kozak sequence and an intervening sequence (intron) flanked by donor and acceptor sites for RNA splicing. Highly efficient transcription initiation can be achieved with the early and late promoters from SV40, the long terminal repeats (LTRs) from retroviruses (e.g., RSV, HTLV I, HIV) and the early promoter of the cytomegalovirus (CMV). However, cellular promoters can also be used (e.g., the human actin promoter). Suitable expression vectors for use in practicing the present invention include, but are not limited to, pIRES1neo, pRetro-Off, pRetro-On, PLXSN, or pLNCX (Clontech), pcDNA3.1 (+/−), pcDNA/Zeo (+/−) or pcDNA3.1/Hygro (+/−) (Invitrogen), pSVL and pMSG (Pharmacia, Uppsala, Sweden), pRSVcat (ATCC 37152), pSV2dhfr (ATCC 37146) and pBC12MI (ATCC 67109). Suitable mammalian host cells include, but are not limited to, human Hela 293 (ATCC CRL-1573), H9 (ATCC HTB-176), Jurkat cells (ATCC CRL-1990), mouse NIH3T3 (ATCC HB1 1601), C127 cells (ATCC CRL-1804), Cos 1, Cos 7, NSO, and CV 1, quail QC1-3 cells, mouse L cells (ATCC CCL-1) and Chinese hamster ovary (CHO) cells (ATCC CCL-61).

Alternatively, the nucleic acid encoding the polypeptide of interest is expressed in stable cell lines that contain the nucleic acid integrated into a host chromosome. The co-transfection of the nucleic acid encoding the polypeptide of interest along with a gene encoding a selectable marker such as, for example, DHRF (dihydrofolate reductase), GPT neomycin, or hygromycin allows the identification and isolation of the transfected cells.

The transfected gene can also be amplified to express large amounts of the encoded polypeptide. The DHFR marker is useful to develop cell lines that carry several hundred or even several thousand copies of the gene of interest. Another useful telection marker is the enzyme glutamine synthase (GS) (Murphy, et al., Biochem. J. 227:277 (1991); Bebbington, et al., BioTechnology 10:169 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong LTR promoter of the Rous Sarcoma Virus (Cullen, et al., Molec. Cell. Biol. 5:438 (1985)) plus a fragment of the CMV-enhancer (Boshart, et al., Cell 41:521 (1985)). Multiple cloning sites (e.g., with the restriction enzyme cleavage sites BamH I, Xba I and Asp 718), facilitate the cloning of the gene of interest. The vectors contain, in addition to the 3′ intron, the polyadenylation and termination signal of the rat preproinsulin gene.

### Example 5

### Overexpression of huJAM Splice Variants in Xenopus Embryos

Injecting RNA that encodes novel proteins into early Xenopus laevis embryos may result in various phenotypes in the early tadpoles (i.e. split axis, anteriorization etc.) that help elucidate the function of these proteins.

The DNA encoding the polypeptide of interest is first cloned into an expression vector (e.g., PR1) containing the T7 RNA polymerase promoter. Plasmid DNA containing the cDNA insert is linearized and in vitro transcribed using T7 RNA polymerase as known in the art. The RNAs produced are examined on a 1.25% agarose gel to confirm success of the transcription reaction as well as the appropriate size and concentration of the RNA product. The RNA is translated in vitro using the biotin in vitro translation kit (Roche, #1559451) to determine if RNA is of sufficient quality to produce protein of predicted molecular weight. Finally, RNA is diluted to 200 μg/ml with Rnase-free water and stored at −20° C. until ready for microinjection into Xenopus embryos.

Female Xenopus frogs are injected with 300 U/frog of human choronic gonadotropin (Sigma Corp., St. Louis, Mo.) to induce egg laying. Eggs are harvested from the females and combined with macerated testis to fertilize the eggs in vitro.

Fertilized eggs are dejellied with 2% cysteine in water (pH 8.0), rinsed, and transferred to 1× MR (0.1 M NaCl, 1.8 nM KCl, 2.0 mM CaCl₂, 1.0 mM MgCl₂ and 50 mM HEPES-NaOH). Embryos develop at room temperature or 15° C. until signs of the first cleavage furrow appear. Embryos are then transferred to 4% Ficoll (Sigma Corp.) in 1× MR prior to injection.

One ng of RNA is injected into both cells of a two-cell stage embryo. Uninjected controls are also maintained. Embryos are left in 4% Ficoll/1× MR solution until they reach the blastula stage of development, then they are switched to 0.1× MR with 4% Ficoll and continue to develop at 18° C. Embryos are observed for morphological effects during the next 2-3 days. Phenotypes of tadpoles are recorded by photography using a digital camera and a dissecting microscope.

Histological analysis of embryos is performed. The tadpoles are fixed in 3% formaldehyde and embedded in paraffin. Tissues are prepared for analysis by removing the parafin with xylene and then gradually rehydrating the tissue with graded solutions of ethanol and water. Sections are then stained with hematoxylin and eosin solutions, coverslipped and viewed under a light microscope.

Animal Cap Assays with huJAMsv Polypeptides

Animal cap explants from early Xenopus embryos normally give rise to cells solely of the ectodermal lineage. Addition or expression of foreign proteins can change the fate of these cells into other lineages (endodermal and mesodermal) thereby helping to functionate newly discovered proteins.

RNA-injected embryos at stage 8 (Niewkoop and Faber, 1967) are harvested for animal cap studies. Vitelline membranes are removed from the embryos and a sheet of cells from the animal pole (animal cap) is isolated. The animal caps are grown in a 96-well tissue culture dish (Costar) with animal cap buffer [0.2×MR, 1% BSA (Sigma Corp.), and 59 μg/ml Gentamicin (Sigma Corp.)] overnight at room temperature until control embryos reach approximately stage 17-19 (Niewkoop and Faber, 1967).

In addition, animal cap assays can also be performed with uninjected embryos with addition of a protein of interest (1-20 nM) to the animal cap buffer.

Results of these assays are monitored by morphology, RT-PCR for tissue specific gene expression, and immunohistochemistry for visualization of tissue type and distribution within the explant.

### Example 6

### Tissue Distribution of huJAMsv mRNA

Non-radioactive northern blot analysis is performed to examine gene expression in human tissues. First, a control probe, Human G3PDH cDNA Control Probe (Clontech #9805-1), is labeled with digoxigenin using DIG-High Prime Labeling kit (Roche #1585606).

The cDNA encoding a polypeptide of interest is labeled and hybridized to, e.g., human multiple tissue northern (MTN) blot membranes (Clontech #7780-1), human cardiovascular system MTN blot (Clontech #7791-1) and human endothelial cells, diseased, and normal heart using, e.g., DIG Easy Hyb (Roche # 1603558). Membranes are prehybridized with DIG Easy Hyb for 1 hour at 50° C. The DIG DNA probes prepared above are denatured at 100° C. for 10 minutes then immediately placed on ice, diluted with DIG Easy Hyb (2 μl probe to 10 ml Easy Hyb), and the membranes are incubated overnight at 50° C. with the diluted hybridization/probe mixture.

Detection of the membranes is accomplished using CDP-Star chemiluminescent substrate (Roche # 1685627) and X-ray film. The membranes are washed twice in 2× SSC at 5° C. for 30 minutes each, twice in 0.5× SSC at 50° C. for 30 minutes each and twice in 0.1× SSC at 50° C. for 30 minutes each. The membranes are blocked with DIG Blocking Buffer (Wash and Block Buffer Kit, Roche # 1585762) for one hour to overnight at room temperature, and anti-digoxigenin-AP Fab fragments (Roche # 1093274) are then added at a 1:20,000 dilution for 2 hours to overnight at room temperature. The membranes are washed three times in 1× wash buffer, equilibrated in detection solution (DIG Wash and Block Buffer Kit, Roche # 1585762) then incubated with CDP-Star (Roche # 1685627) at 1:100 dilution for 5 minutes at room temperature. After lightly blotting, the membranes are placed between two pieces of transparency film and exposed to X-ray film for various exposure times to ensure detection of all possible bands.

Northern blot data indicate that the main tissue of huJAM2 (wild type) expression is the heart and placenta, with lesser amounts present in brain, smooth muscle, kidney, small intesting, and lung tissues and even less still in the colon, thymus, spleen and liver. All areas of the heart express huJAM2. Fetal heart appears to have a more intense signal compared to adult heart. Thus, the DNA encoding extracellular huJAM and their respective mRNA and proteins are contemplated to be useful in the treatment of cardiovascular diseases. In addition, huJAM2 may be involved in inflammatory diseases of the kidney, small intestine, lung, colon and liver.

### Example 7

### Binding Assays

A BIAcore 2000 instrument is used to detect real-time binding between immobilized huJAMsv and a soluble huJAMsv ligand. huJAMsv is diluted to a concentration of 50 μg/mL in 10 mM sodium acetate buffer at pH 5.0. HuJAMsv is immobilized to a CM5 sensor chip using the amine coupling method.

Potential huJAMsv ligands are diluted in HBS-EP buffer. Samples are injected over HuJAMsv and control surfaces using the kinject method. For example, samples containing 5 μg/mL and 1 μg/mL of protein are injected at 30 μL/min for three minutes with a ninety second dissociation time. HuJAMsv and control surfaces are then regenerated with glycine-hydrochloride at pH 3.0.

Protein Binding in Human Tissue

Binding of huJAMsv proteins to human tissues is determined by protein staining with fluorescent dye. All tissues are fixed with 3% paraformaldehyde and embedded in paraffin. Tissues are prepared for analysis by removing the paraffin with xylene then gradually rehydrating the tissue with graded solutions of ethanol and water. Antigen retrieval is performed to unmask antigenic sites so that antibodies can recognize the antigen. This is accomplished by soaking the tissue in citrate buffer (Dako, Carpinteria, Calif.) for twenty minutes at 80 to 90° C. followed ten minutes at ambient temperature. The tissue is then washed in tris-buffered saline (TBS) containing 0.05% TWEEN®-20 and 0.01% thimerosol. To minimize non-specific background staining, the tissue is soaked in non-serum protein block (Dako) for forty-five minutes, after which the protein block is removed by blowing air over the tissue.

The tissue is exposed for two hours to the FLAG-HIS tagged huJAMsv protein at 10 μg/mL. Following exposure, the tissue is washed twice with TBS containing 0.05% TWEEN®-20 and 0.01% thimerosol. The tissue sample is then incubated for one hour with mouse anti-FLAG antibody at 10 μg/mL. Subsequently, the tissue is washed twice with TBS containing 0.05% TWEEN®-20 and 0.01% thimerosol. Next, the tissue is exposed to rabbit anti-mouse Ig with Alexa 568, a fluorescent dye, at 10 μg/mL for one hour, followed again by two washes with TBS containing 0.05% TWEEN®-20 and 0.01% thimerosol. Finally, the tissue is coverslipped with fluorescence mounting media, and the fluorescence is measured. A positive fluorescence reading indicates that the protein binds with antigens on the tissue, suggesting localization of possible ligand or receptor for that polypeptide.

### Example 8

### In Vivo Function with Endotoxin Challenged Mice

Endotoxin is a lipopolysaccharide (LPS) from gram negative bacterial cells which immediately induces systemic release of inflammatory mediators such as TNF and IL1. Mice injected with LPS usually die within 48 hours of injection. DNA injected into the tail veins of mice will translocate to the liver where protein encoded by the DNA will be produced and secreted into the blood stream. Injection of IL-10 DNA protects mice from death when challenged with LPS. Injection of polypeptides of interest will test their anti-inflammatory properties.

Plasmid DNA for huJAMsv under control of the CMV promoter is prepared from DH5-αE. coli cells using an endotoxin free DNA isolation kit (Qiagen). IL-10 is cloned behind a CMV promoter in the pcDNA3.1/v5-His-TOPO vector (Invitrogen #K4800-01) as a positive control DNA (IL-10 abrogates the effects of the endotoxin). DNA is quantified using a spectrophotometer and prepared for injection with the TRANSIT in vivo gene delivery kit (Mirus Corp). Mice (Harlan) aged 6-8 week and weighing on average 24.5 g are injected with 20 ug/mouse (in 2.4 mls) of huJAM2sv1, huJAM3sv1, huJAM3sv2, IL-10 or vector alone DNA into the tail vein. Twenty-four hours after DNA injection, the mice are challenged with endotoxin and D-galactosamine (10 μg/mouse and 6 mg/mouse respectively). Mice are monitored 3× daily for 72 hours to determine survival. Two hours post LPS injection, retro-orbital bleeds are done to collect serum for analysis of cytokines by Luminex (BioRad, #171-F12080).

It is contemplated that the huJAMsv DNA injected into the mice is transcribed then translated into protein and secreted into the blood stream. It is further contemplated that mice injected with huJAMsv that survive the LPS effect, do so by reducing or eliminating the inflammatory response resulting from the LPS.

### Example 9

### Blocking Migration of Immune Cells

This assay serves to determine if a huJAMsv polypeptide (or fusion polypeptide, e.g., fused to an Fc domain) can block migration of immune cells (mainly lymphocytes) from the blood into the peritoneal cavity after puncturing the intestine. A Balb/c mouse is injected with a polypeptide of interest (about 0-10 mg/kg, IV) or with a control polypeptide while the mouse is anesthetized. Cecal Ligation Puncture (CLP) is performed immediately after injection is administered. Naïve mice are used to establish a baseline control. It is preferably that each polypeptide is tested in a group consisting of 10 mice. The mice are sacrificed and samples are collected 24 hours after the CLP. The sample materials are circulating blood and peritoneal wash for cell counts by Hemavet. Peritoneal wash samples may also be used for cytokine analysis. If With treatment of LP10034Fc we would, in the blood, expect the number of white blood cells and lymphocytes to remain constant or does not decrease to about 2000 cells/ul 24 hrs post CLP and/or the lymphocytes from the peritoneal wash remain constant (at around 1500 cells/ul) or do not increase to ˜3000 cells/ul, this would indicate that the polypeptide tested is blocking or reducing the migration of lymphocytes from the blood into the peritoneal cavity.

1. An isolated huJAM splice variant polypeptide comprising a polypeptide with a sequence which is at least 99% identical to a sequence selected from the group consisting of:
a) amino acids 1-323 of SEQ ID NO: 2,
b) amino acids 29-323 of SEQ ID NO: 2,
c) amino acids 1-229 of SEQ ID NO: 4,
d) amino acids 31-229 of SEQ ID NO: 4,
e) amino acids 1-265 of SEQ ID NO: 5,
f) amino acids 31-265 of SEQ ID NO: 5,
g) amino acids 1-242 of SEQ ID NO: 5, and
h) amino acids 31-242 of SEQ ID NO: 5.

2. The isolated polypeptide of claim 1 wherein said polypeptide has a sequence identical to a sequence selected from the group consisting of:
a) amino acids 1-323 of SEQ ID NO: 2,
b) amino acids 29-323 of SEQ ID NO: 2,
c) amino acids 1-229 of SEQ ID NO: 4,
d) amino acids 31-229 of SEQ ID NO: 4,
e) amino acids 1-265 of SEQ ID NO: 5,
f) amino acids 31-265 of SEQ ID NO: 5,
g) amino acids 1-242 of SEQ ID NO: 5, and
h) amino acids 31-242 of SEQ ID NO: 5.

3. The isolated polypeptide of claim 1, wherein said polypeptide is capable of binding a huJAM ligand.

4. An isolated polynucleotide encoding a huJAM splice variant polypeptide comprising a sequence which is at least 99% identical to a sequence selected from the group consisting of:
a) amino acids 1-323 of SEQ ID NO: 2,
b) amino acids 29-323 of SEQ ID NO: 2,
c) amino acids 1-229 of SEQ ID NO: 4,
d) amino acids 31-229 of SEQ ID NO: 4,
e) amino acids 1-265 of SEQ ID NO: 5,
f) amino acids 31-265 of SEQ ID NO: 5,
g) amino acids 1-242 of SEQ ID NO: 5, and
h) amino acids 31-242 of SEQ ID NO: 5.

5. The isolated polynucleotide of claim 4 encoding a huJAM splice variant polypeptide comprising a sequence which is identical to a sequence selected from the group consisting of:
a) amino acids 1-323 of SEQ ID NO: 2,
b) amino acids 29-323 of SEQ ID NO: 2,
c) amino acids 1-229 of SEQ ID NO: 4,
d) amino acids 31-229 of SEQ ID NO: 4,
e) amino acids 1-265 of SEQ ID NO: 5,
f) amino acids 31-265 of SEQ ID NO: 5,
g) amino acids 1-242 of SEQ ID NO: 5, and
h) amino acids 31-242 of SEQ ID NO: 5.

6. An isolated polynucleotide encoding a huJAM splice variant wherein said polynucleotide is selected from the group consisting of SEQ ID NO: 6, SEQ ID NO: 7, and SEQ ID NO: 8.

7. An expression vector comprising the polynucleotide of any of claims 4, 5 or 6.

8. A host cell transfected with the vector of claim 7.

9. The host cell of claim 8, wherein the host cell is selected from the group consisting of mammalian cells, E. coli cells, Sf9 cells and yeast cells.

10. A method for producing the polypeptide of claim 1, the method comprising the steps of:
a) culturing the host cell of claim 8 under conditions suitable for expression of the polypeptide; and
b) recovering the polypeptide from the host cell culture medium.

11. An isolated polypeptide produced by the method of claim 10.

12. A pharmaceutical composition comprising the isolated polypeptide of claim 1 and a pharmaceutically acceptable carrier.

13. A method of treating an immune system disorder comprising administering a therapeutically effective amount of the purified polypeptide of claim 1 to a mammal having said disorder.

14. The method of claim 13, wherein the immune system disorder is an immune deficiency, an autoimmune disease or an inflammatory disorder.

15. A method of treating cancer comprising administering a therapeutically effective amount of the purified polypeptide of claim 1 to a mammal having cancer.

16. A method of treating a cardiovascular disorder comprising administering a therapeutically effective amount of the purified polypeptide of claim 1 to a mammal having said disorder.

17. A method of treating wound healing comprising administering a therapeutically effective amount of the purified polypeptide of claim 1 to a mammal in need of such treatment.
